(19) United States
(12) Patent Application Publication
(10) Pub. No.: US 2015/0010933 A1
(43) Pub. Date: Jan. 8, 2015

(54) Novel Screening Methods

(71) Applicant: The Trustees of the University of Pennsylvania, Philadelphia, PA (US)

(72) Inventors: Harvey Rubin, Philadelphia, PA (US); Takahiro Yano, Havertown, PA (US)

(21) Appl. No.: 14/376,204

(22) PCT Filed: Feb. 13, 2013
(86) PCT No.: PCT/US13/25933

§ 371 Date: Aug. 1, 2014

Related U.S. Application Data
(60) Provisional application No. 61/598,156, filed on Feb. 13, 2012.

(51) Int. Cl. C12Q 1/02; C12Q 1/66

(52) U.S. Cl. CPC C12Q 1/025; C12Q 1/66; G01N 2500/04
USPC 435/8

(57) ABSTRACT

The present invention includes a luciferase-based high-throughput screening assay that identifies compounds that are inhibitors of cellular metabolism. This assay is applicable to all bacterial and eukaryotic membranes.

### BACKGROUND OF THE INVENTION

A crucial characteristic of bacteria is that they develop resistance to antibiotics due to selection pressure and genetic mutation. Therefore, antibiotic development must be a continuous process that generates novel drugs to always stay ahead of the bacteria's ability to develop resistance to current antibiotics. Failure to do so will lead to the emergence and persistence of antibiotic resistant microbes that cause high morbidity and mortality infections, with few or no antibiotic countermeasures.

Indeed this is happening today—increased antibiotic resistance is acutely felt in the form of difficult-to-treat hospital acquired infections (HAIs) caused by extremely antibiotic-resistant bacteria. In this regard, Gram positive organisms including methicillin-resistant Staphylococcus aureus (MRSA) and the Enterococci are of great concern. An equally and perhaps an even more dangerous situation exists with respect to infections caused by Gram negative rods (GNRs) such as Acinetobacter baumannii, Escherichia coli, Pseudomonas aeruginosa, Klebsiella pneumoniae.

There is therefore an urgent need in the art for development of novel screening methods that allow for the identification of new compounds useful in treating bacterial infections. The present invention meets this need.

### BRIEF SUMMARY OF THE INVENTION

The invention includes a method of identifying a compound that inhibits cellular metabolism. The method comprises isolating a membrane from a cell having a functional oxidative phosphorylation pathway. The method further comprises contacting an aliquot of the isolated membrane with a test compound to yield a first mixture. The method further comprises contacting an aliquot of the isolated membrane with a control compound to yield a second mixture. The method further comprises adding luciferase to the first mixture and to the second mixture. The method further comprises measuring the level of luminescence emitted from the first and second mixtures. If the level of luminescence in the first mixture is lower than the level of luminescence in the second mixture, the test compound is identified as inhibiting cellular metabolism.

In one embodiment, the level of luminescence in the first mixture is less than about 70% of the level of luminescence in the second mixture. In another embodiment, the test compound inhibits or disrupts the activity of at least one enzyme in the oxidative phosphorylation pathway in the cell. In yet another embodiment, the test compound inhibits or disrupts the proton motive force (H⁺ gradient) altering the electrochemical gradient across the cellular membrane affecting the proton concentration across the membrane. In yet another embodiment, the test compound inhibits or disrupts the difference in electric potential across the cellular membrane.

In one embodiment, the cell is eukaryotic. In another embodiment, the eukaryotic cell is mammalian. In yet another embodiment, the cell is bacterial. In yet another embodiment, the bacterium is Gram positive. In yet another embodiment, the Gram positive bacterium is selected from the group consisting of Staphylococcus aureus, Mycobacterium smegmatis, Bacillus thuringiensis, Enterococcus, and combinations thereof. In yet another embodiment, the bacterium is Gram negative. In yet another embodiment, the Gram negative bacterium is selected from the group consisting of Acinetobacter baumannii, Pseudomonas aeruginosa, Yersinia enterocolitica, Escherichia coli, Klebsiella pneumonia, and combinations thereof.

In one embodiment, the luciferase is added at the beginning of the assay. In another embodiment, the assay is carried out over a period of time of two hours or less. In yet another embodiment, the volume of assay reactions ranges from about 5 μL to about 20 μL.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a flowchart illustrating the oxidative phosphorylation (OX/PHOS) system in a Gram negative bacterium.

FIG. 2 is a flowchart illustrating the modular organization of the electron transport chain of the class A, B and C Gram positive bacteria.

FIG. 3 is a graph illustrating a non-limiting optimization of the assay of the invention.

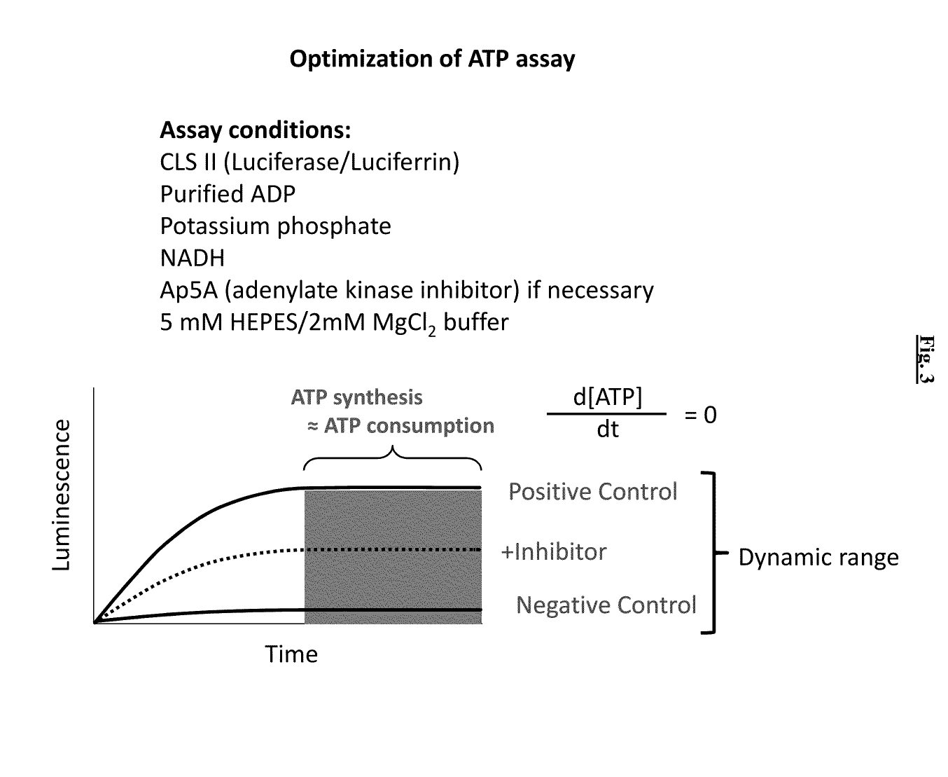

FIG. 4 is a graph illustrating the time course of the ATP synthesis assay for M. smegmatis membrane using 10 μl of reaction volume. In this assay, all reagents, including luciferase/luciferin, were mixed when the reaction started, and the reaction was monitored continuously. In this assay, luminescence activity reached its plateau in ˜15 minutes and remained constant for around 1 hour. Other assays generally require additional steps such as termination of the reaction, addition of luciferase/luciferin, and immediate measurement of luminescence activity. The results indicated that the present assay is amenable to use in high-throughput screening (HTS), in which multiple plates have to be screened in a given run. The present assay afforded a long-lived steady state, which affords a wider time window for measuring the assay readout and thus allows for preparing and reading more plates than other assays.

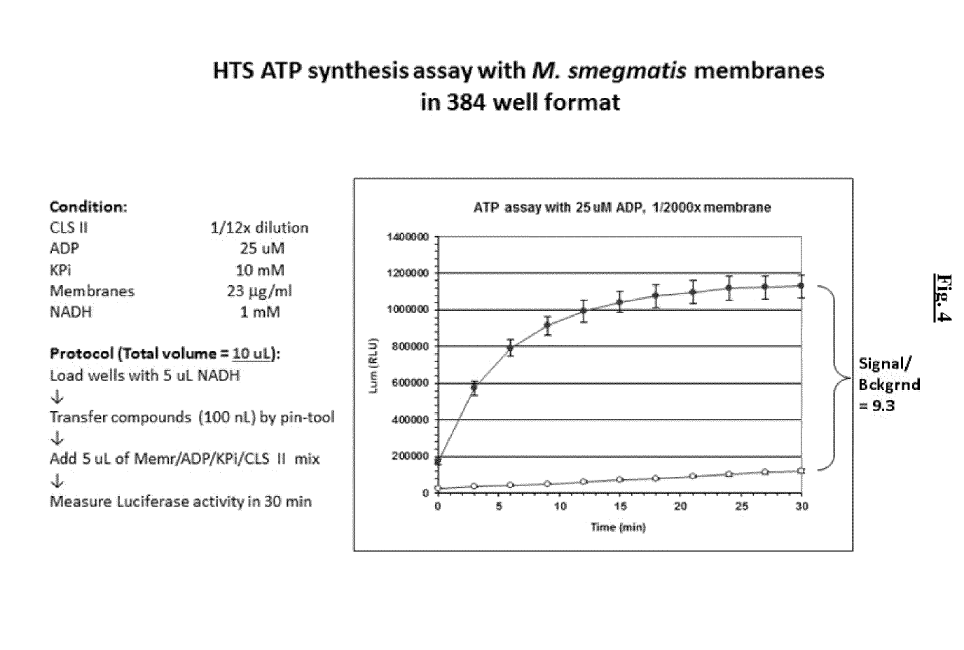

FIG. 5 is a set of graphs illustrating statistic data of ATP synthesis assay with M. smegmatis membranes for 181 microplate assays. The TB alliance library, containing 60,000 compounds, was used here. This data set shows the robustness of the assay, with a high Z-score average of 0.628 and signal-to-background ratios higher than 10.

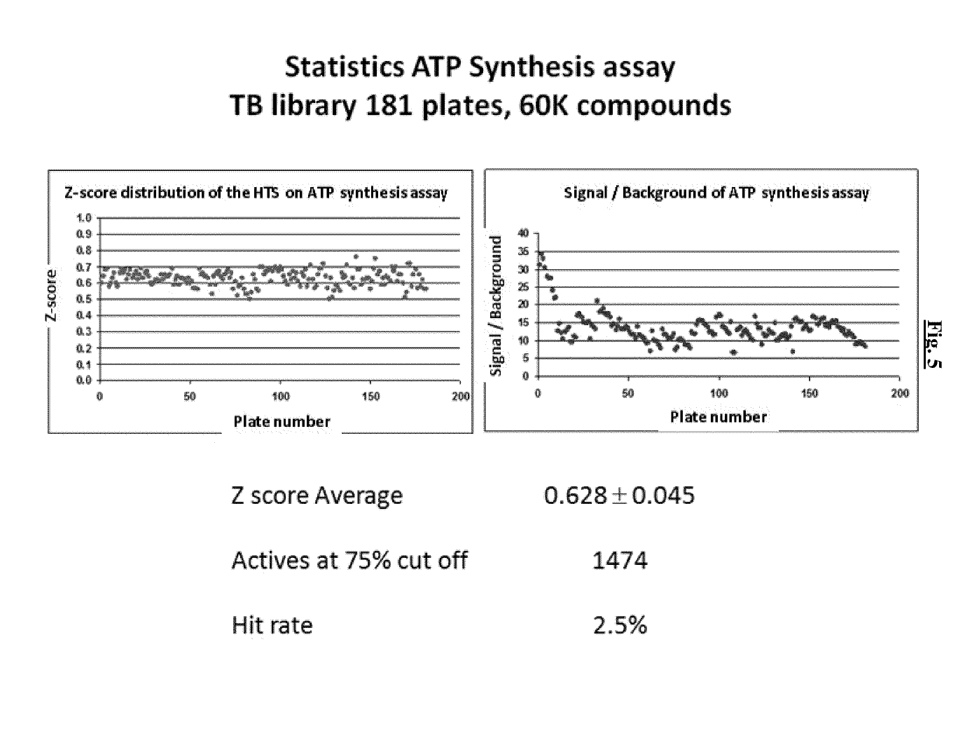

FIG. 6 is a graph illustrating a typical result of HTS. The assay identifies compounds that inhibit OxPhos activity of M. smegmatis. The lower line represents a cut-off line with 75% inhibition in this case.

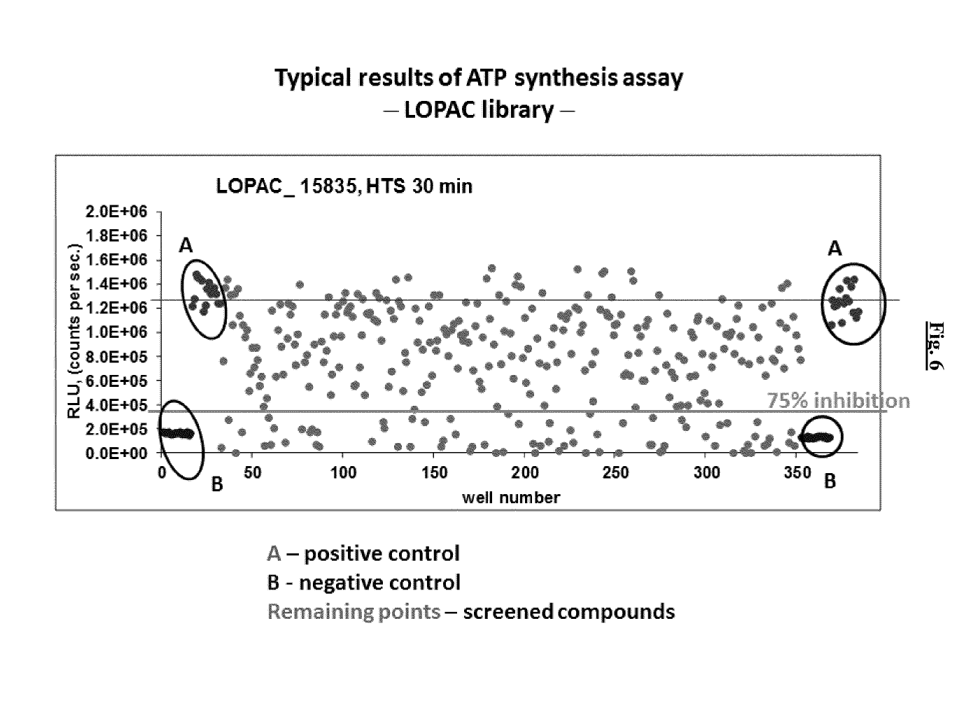

FIG. 7 is a table illustrating Z-scores and S/B ratios for the screening with eight different bacterial membranes. M. smegmatis, Bacillus thuringiensis and Staphylococcus aureus are Gram positive bacteria, and Yersinia enterocolitica, Pseudomonas aeruginosa, Acinetobacter baumannii and Escherichia coli are Gram negative bacteria. In these three bacterial screenings, Z-scores higher than 0.6 with wide dynamic ranges were observed. These results demonstrated that the assay may be readily applied to different bacteria, producing significantly high statistic results.

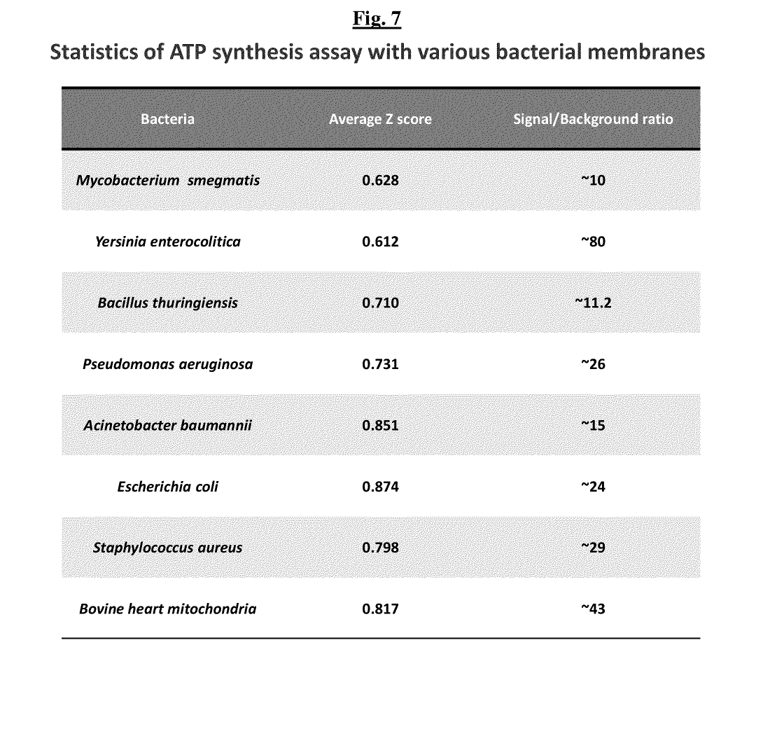

FIG. 8 is a schematic representation of the anti-tuberculosis drug TMC207 or 8207910 [(1R,2S)-1-(6-bromo-2-methoxy-3-quinolyl)-4-dimethylamino-2-(1-naphthyl)-1-phenyl-butan-2-ol], which specifically inhibits ATP synthase of M. tuberculosis.

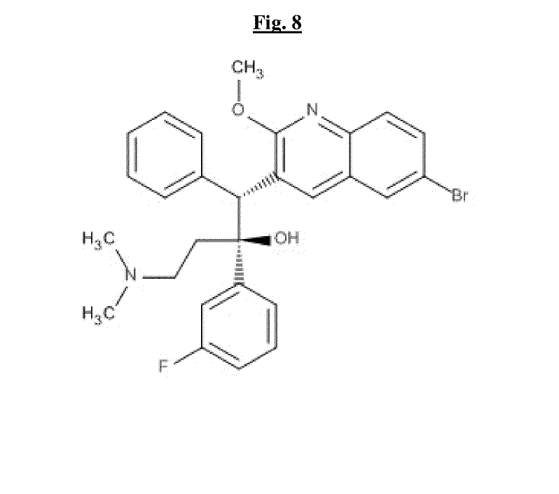

FIG. 9 is a graph illustrating the effect of TMC207 on ATP synthesis activity of M. smegmatis membranes. The assay was carried out in a 384 well plate. The reaction (a total volume 20 μL) contained 1/12×CLSII, 1 mM NADH, 25 μM ADP, 10 mM K-Pi, and 10 μg/ml M. smegmatis membrane in 5 mM Heps/2 mM MgCl₂ buffer (pH 7.0). ATP synthesis activity was normalized to the positive control and IC₅₀ value was determined to be 0.147 nM.

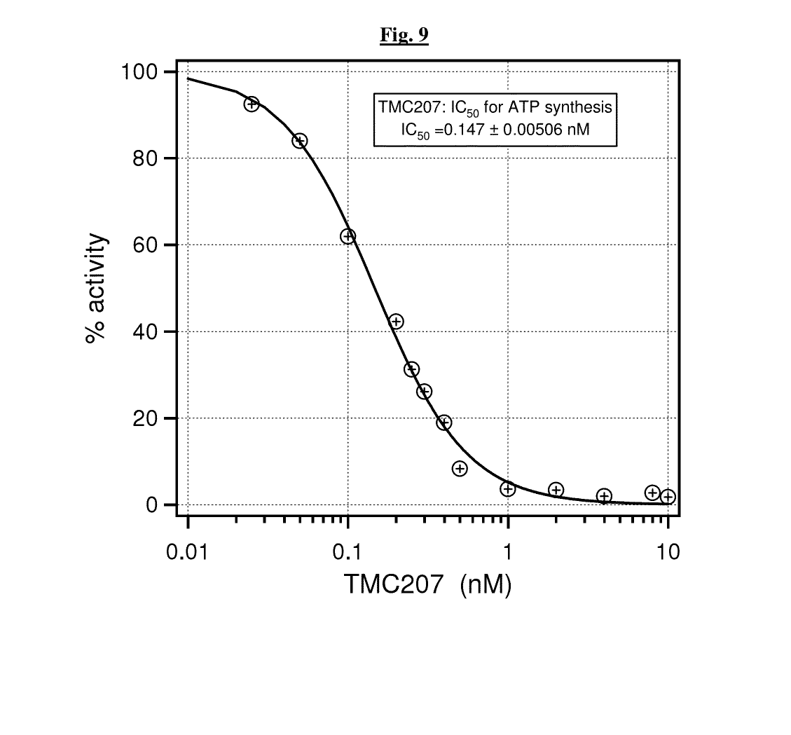

FIG. 10 is a graph illustrating the effect of TMC207 on the NADH oxidase activity of M. smegmatis membranes. The assay was carried out in the presence of various concentrations of TMC207. The reactions contained 0.2 mM NADH and 10 μg/ml membrane in 50 mM Hepes/2 mM MgCl₂ (pH 7.0) buffer. TMC207 did not inhibit the NADH oxidase activity at 10 μM. Only marginal inhibition was observed at only high concentrations.

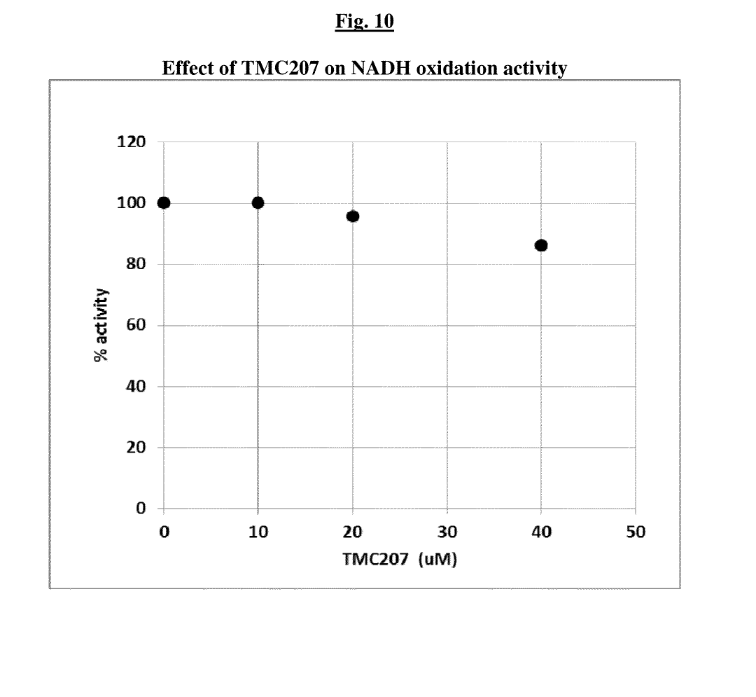

FIG. 11 is a graph illustrating the effect of TMC207 on ΔpH across the membranes. ΔpH was monitored by using a pH sensitive fluorescent dye 9-amino-6-chloro-2-methoxyaridine (ACMA). The reactions contained 1 μM ACMA, 10 μg/ml M. smegmatis membrane in 5 mM Hepes/2 mM MgCl₂ buffer (pH 7.0). The reaction was initiated by addition of 1 mM NADH (open symbols) and NADH was omitted in negative controls (closed symbols). Fluorescence was measured by a plate reader with 420 nm for excitation and 510 nm for emission wavelength. TMC207 did not affect the ΔpH across the membrane.

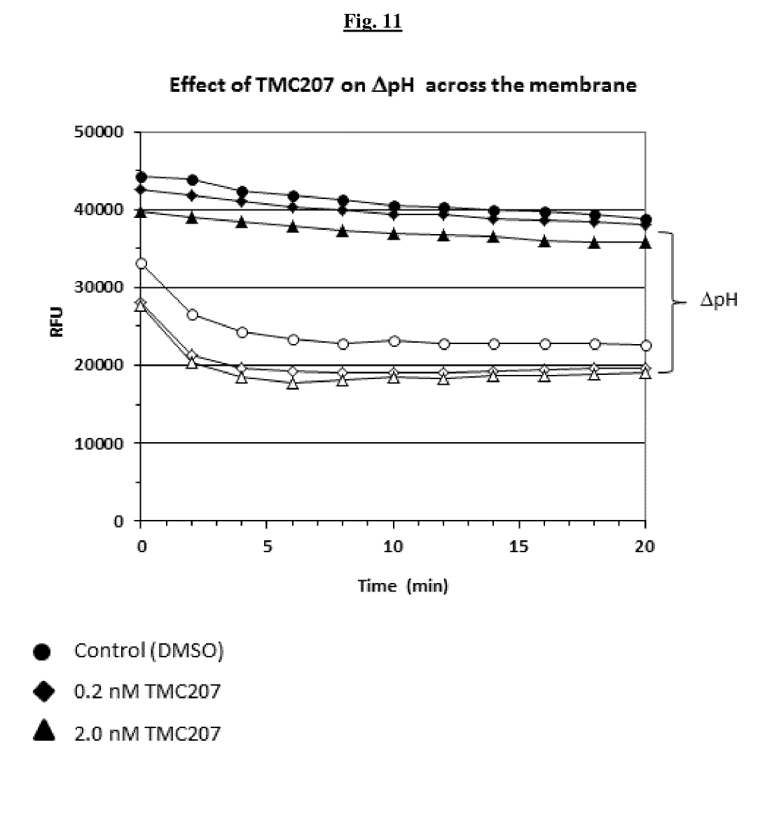

### DETAILED DESCRIPTION OF THE INVENTION

The present invention is related to novel screening methods for compounds useful in the inhibition of cellular metabolism. The present invention includes a novel, high-throughput, luciferase-based assay for identifying compounds that inhibit cellular metabolism. The present invention further includes compounds identified as inhibitory compounds using the methods of the invention.

In one embodiment, an inhibitory compound identified by the method of the present invention inhibits or otherwise disrupts the activity of the enzymes in the oxidative phosphorylation pathway in a Gram-positive or Gram-negative bacterium.

In one embodiment, the assay of the invention is used as a counter assay for compounds to be administered to mammals, preferentially humans.

In one embodiment, an inhibitory compound identified by the method of the present invention inhibits or otherwise disrupts the proton motive force (H⁺ gradient) altering the electrochemical gradient across the cellular membrane affecting the proton concentration across the membrane.

In one embodiment, an inhibitory compound identified by the method of the present invention inhibits or otherwise disrupts the difference in electric potential across the cellular membrane.

In one embodiment, an inhibitory compound identified by the methods of the invention disclosed herein is useful in treating a metabolic disease in a mammal in need thereof.

In one embodiment, an inhibitory compound identified by the methods of the invention disclosed herein is useful in treating a bacterial infection in a mammal in need thereof.

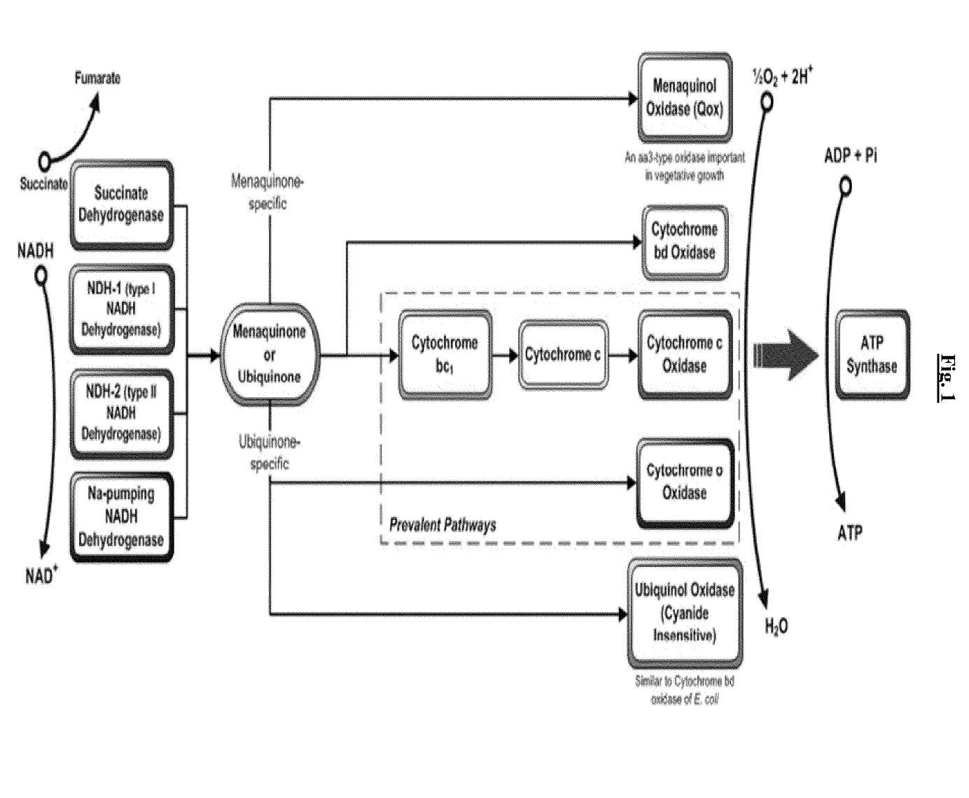

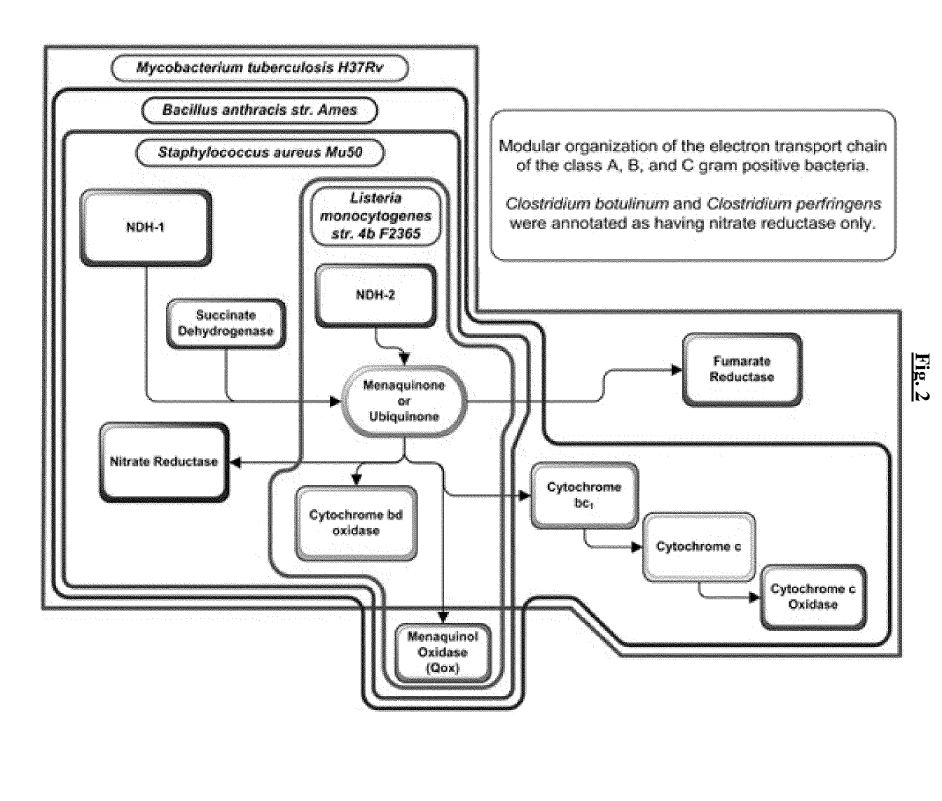

In one aspect, the methods of the invention allow for the discovery and development of new antibiotics against Gram negative and Gram positive bacteria by focusing on identifying inhibitors of the enzymes comprising the energy production pathway. The oxidative phosphorylation (OxPhos) pathway consists of the ATP synthase and Electron Transport Chain (ETC) (FIGS. 1 and 2).

The high-throughput screening (HTS) assay of bacterial membranes with a functional OxPhos pathway allows for the identification of new compounds that are selective inhibitors of bacteria, including but not limited to: Staphylococcus aureus, Enterococcus, Acinetobacter baumannii, Pseudomonas aeruginosa, Escherichia coli, Klebsiella pneumonia, and combinations thereof.

In a non-limiting embodiment, the OxPhos pathways of Gram negative or positive organisms and the human mitochondrial OxPhos pathway are distinct. In another non-limiting embodiment, the HTS assays are performed using isolated OxPhos active Gram negative and Gram positive bacterial membranes, allowing for identification of hit compounds. Selected hit compounds may then be analyzed in bacterial and mammalian cell growth assays, well established animal models of infection, and mechanism of action assays. Further, these hit compounds may be chemically modified to improve their activity and/or developability.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “treatment” means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

### Methods

The invention includes a novel luciferase-based high throughput screening (HTS) assay for identifying compounds that inhibit cellular metabolism. The assay is based on an end point detection of bioluminescence by the luciferin-luciferase system measuring the ATP production activity of the oxidative phosphorylation of membrane vesicles. The assay comprises screening at least one test compound, in the presence of at least one control, in order to evaluate the efficacy of the test compound in inhibiting the oxidative phosphorylation pathway in isolated cellular membranes. This assay does not have to be stopped to add the readout reagents (luciferin-luciferase) and is carried out over an extended period of time (up to two hours) allowing reliable, reproducible dynamic range of measurements. This unique assay is thus highly suitable for high throughput screening (HTS). In one embodiment, the assay affords stable endpoints (steady state) for an extended period of time. The assay is applicable to all bacterial and eukaryotic membranes. Sites of action that could be discovered using this assay include all enzymes in the OxPhos pathway and proton motive force inhibitors which disrupt the electrochemical gradient across the membrane altering either the proton concentration (H+ gradient) and/or the difference in electrochemical potential across the membrane.

The assay is general, using membranes prepared from bacteria as well as eukaryotes, including mammalian cells. Further, the assay is robust, measuring steady state concentrations of ATP produced by these membranes and not relying on more unreliable single point or initial reaction velocity measurements. The assay has immediate utility in high throughput screening format, and may be run in a reaction volume ranging from about 5 to about 20 microliters. In a non-limiting embodiment, the reaction volume is about 10 microliters. The assay is sensitive, detecting inhibitors of ATP synthesis at submicromolar concentrations. The assay is reproducible, with a Z-score of 0.5 or higher.

The assay comprises the steps of:

(a) isolating a membrane from cells;
(b) contacting a cellular membrane with a test compound;
(c) quantifying the luciferase marker in the cellular membrane contacted with the test compound and comparing the amount of detector observed in the presence of a test compound to the amount of gene marker detected in a control.

It will be readily understood by the skilled artisan that more than one test compound may be screened at a time in a given assay. In one embodiment, at least one test compound is screened in an assay. In another embodiment, between about 1 and about 10 test compounds are screened in an assay. In yet another embodiment, between about 1 and about 100 test compounds are screened in an assay.

In yet another embodiment, between about 1 and about 1,000 test compounds are screened in an assay. In yet another embodiment, between about 1 and about 5,000 test compounds are screened in an assay. In still another embodiment, between about 1 and about 10,000 test compounds are screened in an assay.

In one embodiment, the luminescence resulting from the reaction of luciferase with its substrate is measured using techniques well known in the art. In one embodiment, if a test compound inhibits the luminescence resulting from the reaction of a luciferase with its substrate by greater than or equal to 70% of the luminescence of a control, the test compound is identified as an inhibitory compound.

In one embodiment, a control is used to determine if the test compound is an inhibitor of luciferase. In another embodiment, at least one control is run for each assay, regardless of the number of test compounds being evaluated in a given assay. In yet another embodiment, a control used in the assay is a compound or composition known not to inhibit the production of ATP in the cellular membrane. Accordingly, a control in the instant assay may comprise a vehicle, such as a buffer, solution, or medium, used for the preparation of a test compound where the vehicle does not further comprise a test drug. A control in the assay may also comprise a compound known not to be an inhibitory compound.

The control is measured for luminescence resulting from the reaction of luciferase and its substrate at the same time as the luminescence for test compounds is quantified. The luminescence measurements for a test compound and a control are compared and the percent inhibition is calculated for a test compound relative to a control.

Test molecules for use in the present method of identifying an inhibitory compound may be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145). Examples of methods for the synthesis of molecular libraries are known in the literature and may be appropriately modified or adapted by those skilled in the art.

Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869), or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J Mol. Biol. 222:301-310).

The resulting libraries of candidate molecules may be screened using the assay described herein for efficacy in inhibiting cellular metabolism.

### Compositions

The inhibitory compounds of the present invention, which inhibit cellular metabolism, comprise compounds identified using the assays described herein. In one embodiment, a compound identified by an assay of the invention is useful in treating bacterial infection in a mammal, preferably a human. A compound identified by a method of the present invention may be a peptide, a nucleic acid, a small molecule, or other drug that inhibits cellular metabolism. Accordingly, the compounds identified by the methods of the present invention are not limited to those recited specifically herein.

### Nucleic Acids

When the inhibitory compound identified by the method of the present invention comprises a nucleic acid, any number of procedures may be used for the generation of an isolated nucleic acid encoding the compound as well as derivative or variant forms of the isolated nucleic acid, including using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) or direct synthesis of the nucleic acid. For recombinant nucleic acids encoding the compound and in vitro transcription, DNA encoding RNA molecules can be obtained from known clones of the compound, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al. 2001, Molecular Cloning: A Laboratory Manual, Cold Spring Harbor Laboratory Press, New York.

### Peptides

When the inhibitory compound identified by the method of the present invention is a peptide, the peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes. Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York.

Peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### Small Molecules

When the inhibitory compound identified by the method of the present invention is a small molecule, the small molecule may be obtained using standard methods known to the skilled artisan. Such methods include chemical organic synthesis or biological means. Biological means include purification from a biological source, recombinant synthesis and in vitro translation systems, using methods well known in the art.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying a compound that inhibits cellular metabolism, the method comprising the steps of:
(a) isolating a membrane from a cell having a functional oxidative phosphorylation pathway;
(b) contacting an aliquot of the isolated membrane with a test compound to yield a first mixture;
(c) contacting an aliquot of the isolated membrane with a control compound to yield a second mixture;
(d) adding luciferase to the first mixture and to the second mixture;
(e) measuring the level of luminescence emitted from the first and second mixtures,
wherein, if the level of luminescence in the first mixture is lower than the level of luminescence in the second mixture, the test compound is identified as inhibiting cellular metabolism.

2. The method of claim 1, wherein the level of luminescence in the first mixture is less than about 70% of the level of luminescence in the second mixture.

3. The method of claim 1, wherein the test compound inhibits or disrupts the activity of at least one enzyme in the oxidative phosphorylation pathway in the cell.

4. The method of claim 1, wherein the test compound inhibits or disrupts the proton motive force (H⁺ gradient) altering the electrochemical gradient across the cellular membrane affecting the proton concentration across the membrane.

5. The method of claim 1, wherein the test compound inhibits or disrupts the difference in electric potential across the cellular membrane.

6. The method of claim 1, wherein the cell is eukaryotic.

7. The method of claim 6, wherein the eukaryotic cell is mammalian.

8. The method of claim 1, wherein the cell is bacterial.

9. The method of claim 8, wherein the bacterium is Gram positive.

10. The method of claim 9, wherein the Gram positive bacterium is selected from the group consisting of Staphylococcus aureus, Mycobacterium smegmatis, Bacillus thuringiensis, Enterococcus, and combinations thereof.

11. The method of claim 8, wherein the bacterium is Gram negative.

12. The method of claim 11, wherein the Gram negative bacterium is selected from the group consisting of Acinetobacter baumannii, Pseudomonas aeruginosa, Yersinia enterocolitica, Escherichia coli, Klebsiella pneumonia, and combinations thereof.

13. The method of claim 1, wherein the luciferase is added at the beginning of the assay.

14. The method of claim 1, wherein the assay is carried out over a period of time of two hours or less.

15. The method of claim 1, wherein the volume of assay reactions ranges from about 5 μL to about 20 μL.
